(19) United States
(12) Patent Application Publication
(10) Pub. No.: US 2016/0243044 A1
(43) Pub. Date: Aug. 25, 2016

(54) SOFT GELATIN CAPSULES CONTAINING FEXOFENADINE

(71) Applicant: Enspire Group LLC, South Plainfield, NJ (US)

(72) Inventors: Abdul Rashid, Livingston, NJ (US); Dahai Guo, Belle Mead, NJ (US); Minh Tran, Secaucus, NJ (US); Zhang Julia Zhang, Scotch Plains, NJ (US)

(73) Assignee: ENSPIRE GROUP LLC, South Plainfield, NJ (US)

(21) Appl. No.: 14/627,589

(22) Filed: Feb. 20, 2015

(51) Int. Cl. A61K 9/48; A61K 31/445

(52) U.S. Cl. CPC A61K 9/4866; A61K 31/445; A61K 9/4858; A61K 9/4825; A61K 9/4891

(57) ABSTRACT

Bioavailable liquid softgel fill compositions comprising a) fexofenadine or a fexofenadine salt; b) a matrix comprising a pharmaceutically acceptable poly(alkylene glycol), a pharmaceutically acceptable alkylene glycol, a pharmaceutically acceptable polymeric solubilizing agent, and a pharmaceutically acceptable surfactant; and c) a pharmaceutically acceptable acidulant are disclosed. Also disclosed are methods for the preparation of such fill compositions, and softgel capsules containing the bioavailable liquid fill composition.

### FIELD OF THE DISCLOSED SUBJECT MATTER

The presently disclosed subject matter relates to bioavailable fill compositions containing a fexofenadine, soft gelatin capsules filled with the bioavailable fexofenadine fill compositions, and methods of making same.

### BACKGROUND

Fexofenadine is an antihistamine pharmaceutical drug used in the treatment of allergy symptoms, such as hay fever, nasal congestion, and urticaria or hives. It is known as a third-generation antihistamine, which means that its effect are limited to the periphery, that is, outside the brain and spinal cord, which is where most antihistamines mediate their sedating effects; therefore, fexofenadine has minimal sedation side effects. Fexofenadine is used for relief from physical symptoms associated with seasonal allergic rhinitis and for treatment of chronic urticaria. It does not cure, but rather prevents the aggravation of rhinitis and urticaria, and reduces the severity of the symptoms associated with those conditions, thereby providing relief from repeated sneezing, runny nose, itchy eyes and general body fatigue.

Fexofenadine is a lipophilic solid which is frequently administered as the hydrochloride salt.

Both fexofenadine free base and fexofenadine salts have poor solubility in aqueous solution, and present difficult problems in formulating for effective administration with adequate bioavailability. A well-designed formulation should, at a minimum, be capable of presenting a therapeutically effective amount of a hydrophobic compound to its desired absorption site, in an absorbable form. Even this minimal functionality is difficult to achieve when delivery of the hydrophobic therapeutic agent requires interaction with aqueous physiological environments, such as gastric and intestinal fluids. Furthermore, drug absorption in various individuals might differ significantly due to differences in gastrointestinal function and food intake. Therefore, it is rather difficult to determine and control the dosage. An additional challenge in the formulation of fexofenadine for oral administration is the low solubility of the compound, especially under the acidic aqueous gastric conditions (solubility of 0.2 mg of fexofenadine HCl per ml of pH 1.2 aqueous buffer solution).

Another issue in the formulation of fexofenadine into oral pharmaceutical compositions is its unpleasant, strong and bitter taste as well as aftertaste, which has led to poor compliance or even non-compliance with the treatment and thus has a negative impact on the efficiency of treatment.

This combination of physical properties requires careful selection of pharmaceutical formulations when formulating fexofenadine for oral administration. There continues to be a need for superior formulations which stabilize and isolate fexofenadine, provide adequate bioabsorption, and ultimately deliver the active ingredient to the appropriate target within the human body.

### SUMMARY OF THE DISCLOSED SUBJECT MATTER

A new formulation providing desirable protection and stabilization of the fexofenadine active ingredient is provided herein. This formulation also provides enhanced bioavailability.

In view of the above considerations, an overall approach to formulating fexofenadine hydrochloride included isolating the active ingredient (AI) within a soft gelatin capsule (softgel) and specifically solubilizing the AI in a suitable fill composition matrix comprising a polymeric solubilizing agent in combination with a surfactant in an alkylene glycol and poly(alkylene glycol) vehicle. The pH of the fill composition is maintained in the acid range with a pharmaceutically acceptable acidulant in a small weight percentage of water.

The solubilizing matrix comprises two parts. Part A is a hydrophilic mixture of a pharmaceutically acceptable alkylene glycol, such as propylene glycol, a pharmaceutically acceptable poly(alkylene glycol), such as a polyethylene glycol (PEG), and a pharmaceutically acceptable solubilizing polymer, such as a polyvinylpyrrolidone (povidone). Part B is a mixture of a pharmaceutically acceptable surfactant, such as sodium lauryl sulfate (SLS), water, and a pharmaceutically acceptable acidulant, such as citric acid. The surfactant can be ionic or non-ionic (e.g. a polysorbate). Parts A and B are combined to form the solubilizing matrix, and fexofenadine hydrochloride is added to this solubilizing matrix to form the fill composition.

The fexofenadine fill composition of the invention can be encapsulated into soft gelatin capsules of the invention.

In some embodiments of the fill formulation, the fexofenadine AI is in the form of fexofenadine free base. In some embodiments the fexofenadine AI is a fexofenadine salt. In one embodiment the fexofenadine salt is fexofenadine hydrochloride. In some embodiments the fexofenadine free base or fexofenadine salt is the only active ingredient. In certain embodiments, one or more additional active ingredients are added to the fexofenadine-containing fill formulation.

One aspect of the invention is directed to a bioavailable liquid softgel fill composition comprising: a) 4-40% by weight of fexofenadine or a fexofenadine salt; b) a matrix comprising: bi) 40-80% by weight of a pharmaceutically acceptable poly(alkylene glycol); bii) 5-30% by weight of a pharmaceutically acceptable alkylene glycol; biii) 1-10% by weight of a pharmaceutically acceptable polymeric solubilizing agent; and biv) 0.001-6% by weight of a pharmaceutically acceptable surfactant; and c) 0.001-2% by weight of a pharmaceutically acceptable acidulant, based on the total weight of the composition. Embodiments of the invention include compositions which have at least one of the following features: (a) the fexofenadine or fexofenadine salt and the matrix are present in a ratio of about 1:1.5 to about 1:24 by weight; or (b) the fexofenadine or fexofenadine salt and the pharmaceutically acceptable polymer are present in a ratio of about 40:1 to about 2:5 by weight; or (c) the fexofenadine or fexofenadine salt and the pharmaceutically acceptable surfactant are present in a ratio of about 40000:1 to about 2:3 by weight. One embodiment of the composition has all of the features (a), (b) and (c). In one embodiment the fexofenadine salt is fexofenadine hydrochloride. In a preferred embodiment the fexofenadine or fexofenadine salt is the only active ingredient. Other embodiments of the composition further comprise one or more additional active ingredients. In one embodiment the pharmaceutically acceptable poly(alkylene glycol) is selected from the group consisting of poly(ethylene glycol)s (PEGs); preferably the PEGs are selected from the group consisting of PEG 200, 300, 400, 600, mixtures thereof, and mixtures of these with PEG 800, 1000, 2000, 3000, 4000, 5000, 6000, 7000, or 8000. In one embodiment the pharmaceutically acceptable alkylene glycol is propylene glycol. In one embodiment of the fill composition, the pharmaceutically acceptable polymeric solubilizing agent is a polyvinylpyrrolidone (PVP). In one embodiment the PVP is selected from the group consisting of PVP K12, PVP K17, PVP K30, PVP K60, and PVP K90; preferably the polyvinylpyrrolidone is PVP K17. In one embodiment the pharmaceutically acceptable surfactant is selected from the group consisting of sodium lauryl sulfate, polysorbates, and PEG-8 caprylic/capric glycerides. In one embodiment the acidulant is selected from the group consisting of pharmaceutically acceptable organic acids and mixtures of two or more thereof. In a preferred embodiment the acidulant is selected from the group consisting of lactic acid, malic acid, citric acid, fumaric acid, ascorbic acid, tartaric acid and mixtures of two or more thereof. In a particularly preferred embodiment the acidulant comprises citric acid.

Another aspect of the invention is directed to a method of preparing the above bioavailable liquid softgel fill composition, comprising the steps of: (a) combining the poly(alkylene glycol) and the alkylene glycol in a stainless steel container and heating the mixture to a temperature of 65±5° C. with stirring for a first period of time to obtain a first mixture; (b) slowly adding the polymeric solubilizing agent in small quantities into the first suspension with stirring and continue stirring for a second period of time after powder addition is complete, at the same temperature to obtain a second mixture; (c) preparing a third mixture by combining the surfactant and acidulant with water in a separate stainless steel container and heating the mixture to a temperature of 65±5° C. with stirring for a third period of time; (d) combining the second and third mixtures at the same temperature with mixing for a fourth period of time to provide a fourth mixture; (e) adding the fexofenadine or fexofenadine salt to the fourth mixture in small quantities with stirring at the same temperature and continuing stirring for a fifth period of time after powder addition has been completed to obtain a fifth mixture; and (f) cooling and deaerating the fifth mixture to ambient temperature, providing the liquid softgel fill composition.

Another aspect of the invention is directed to a softgel capsule comprising a soft gelatin capsule filled with the bioavailable liquid softgel fill composition disclosed above. In one embodiment the gelatin of said soft gelatin (softgel) capsule comprises bovine-, avian-, porcine-, marine- or vegetable-based gelatin, or a mixture of two or more thereof. In one embodiment, the softgel capsule further comprises an enteric coating. The enteric coating preferably comprises a controlled release polymer. In one embodiment the controlled release polymer is an acid-resistant polymer.

Another aspect of the invention is directed to a bioavailable liquid softgel fill composition consisting essentially of: a) 4-40% by weight of fexofenadine hydrochloride; b) a matrix comprising: bi) 40-80% by weight of PEG 400; bii) 5-30% by weight of propylene glycol; biii) 1-10% by weight of polyvinylpyrrolidone; and biv) 0.001-6% by weight of sodium lauryl sulfate; c) 0.001-2% by weight of citric acid; and d) 1-10% of water, based on the total weight of the composition.

Yet another aspect of the invention is directed to a bioavailable liquid softgel fill composition consisting essentially of: a) about 13.9% by weight of fexofenadine hydrochloride; b) a matrix comprising: bi) about 64.2% by weight of PEG 400; bii) about 15.2% by weight of propylene glycol; biii) about 3.4% by weight of polyvinylpyrrolidone; and biv) about 0.3% by weight of sodium lauryl sulfate; c) about 0.5% by weight of citric acid; and d) about 2.5% of water, based on the total weight of the composition.

One embodiment of the above compositions is directed to a softgel capsule comprising a soft gelatin capsule filled with the above bioavailable liquid softgel fill composition. Preferably the polyvinylpyrrolidone is PVP K17.

### DETAILED DESCRIPTION OF PREFERRED EMBODIMENTS

Fexofenadine free base has the structure of Formula (I):

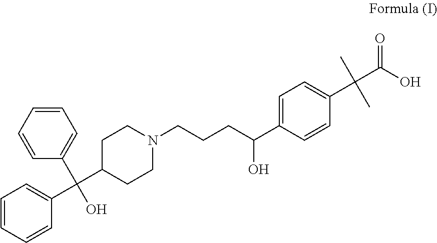 [SMILES: CC(C)(C(=O)O)c1ccc(C(O)CCCN2CCC(C(O)(c3ccccc3)c3ccccc3)CC2)cc1]

A pharmaceutically acceptable salt, such as the hydrochloride salt, rather than the free base is frequently used as the active ingredient in pharmaceutical compositions.

One aspect of the invention is directed to a bioavailable liquid softgel fill composition comprising: a) 4-40% (e.g. 5-35%, 5-20%, 10-30%, 15-25%, about 13% about 14%, about 15% or about 20%) by weight of fexofenadine or a fexofenadine salt; b) a matrix comprising: bi) 40-80% (e.g. 45-75%, 50-70%, 55-65%, about 60%, about 64%, 64-65% or about 65%) by weight of a pharmaceutically acceptable poly(alkylene glycol); bii) 5-30% (e.g. 5-25%, 10-25%, 15-16%, 15-20%, about 14%, about 15% or about 16%) by weight of a pharmaceutically acceptable alkylene glycol; biii) 1-10% (e.g. 1-9%, 2-8%, 3-7%, 4-6%, 3-4%, about 3%, about 4% or about 5%) by weight of a pharmaceutically acceptable polymeric solubilizing agent; and biv) 0.001-6% (e.g., 0.01-2%, 0.1-1%, 0.2-0.5%, about 0.2%, about 0.3% or about 0.4%) by weight of a pharmaceutically acceptable surfactant; and c) 0.001-2% (e.g. 0.01-1%, 0.1-0.8%, 0.4-0.6%, about 0.4%, about 0.5% or about 0.6%) by weight of a pharmaceutically acceptable acidulant, based on the total weight of the composition. Embodiments of the invention include compositions which have at least one of the following features: (a) the fexofenadine or fexofenadine salt and the matrix are present in a ratio of about 1:1.5 to about 1:24 by weight (e.g., about 1:20, about 1:15, about 1:10, about 1:6, about 1:5.7, about 1:5 or about 1:4); or (b) the fexofenadine or fexofenadine salt and the pharmaceutically acceptable polymer are present in a ratio of about 40:1 to about 2:5 by weight (e.g., about 30:1, about 20:1, about 10:1, about 5:1, about 4.1:1, about 4:1 or about 3:1); or (c) the fexofenadine or fexofenadine salt and the pharmaceutically acceptable surfactant are present in a ratio of about 40000:1 to about 2:3 by weight (e.g., about 10000:1, about 5000:1, about 2500:1, about 1000:1, about 500:1, about 100:1, about 50:1, about 46:1, about 40:1, about 30:1, about 20:1 or about 10:1). One embodiment of the composition has all of the features (a), (b) and (c). In one embodiment the fexofenadine salt is fexofenadine hydrochloride. In a preferred embodiment the fexofenadine or fexofenadine salt is the only active ingredient. Other embodiments of the composition further comprise one or more additional active ingredients. In one embodiment the pharmaceutically acceptable poly(alkylene glycol) is selected from the group consisting of poly(ethylene glycol)s (PEGs); preferably the PEGs are selected from the group consisting of PEG 200, 300, 400, 600, mixtures thereof, and mixtures of these with PEG 800, 1000, 2000, 3000, 4000, 5000, 6000, 7000, or 8000. In one embodiment the pharmaceutically acceptable alkylene glycol is propylene glycol. In one embodiment of the fill composition, the pharmaceutically acceptable polymeric solubilizing agent is a polyvinylpyrrolidone (PVP). In one embodiment the PVP is selected from the group consisting of PVP K12, PVP K17, PVP K30, PVP K60, and PVP K90, covering a molecular weight range of about 2,000 to about 1,500,000; preferably the polyvinylpyrrolidone is PVP K17. In one embodiment the pharmaceutically acceptable surfactant is selected from the group consisting of sodium lauryl sulfate, polysorbates (e.g. TWEEN® 20, 40, 60, 80, etc.), and PEG-8 caprylic/capric glycerides (e.g LABRASOL®, also known as caprylocaproyl polyoxyl-8 glycerides). In one embodiment the acidulant is selected from the group consisting of pharmaceutically acceptable organic acids and mixtures of two or more thereof. In a preferred embodiment the acidulant is selected from the group consisting of lactic acid, malic acid, citric acid, fumaric acid, ascorbic acid, tartaric acid and mixtures of two or more thereof. In a particularly preferred embodiment the acidulant comprises citric acid.

Another aspect of the invention is directed to a method of preparing the above bioavailable liquid softgel fill composition, comprising the steps of: (a) combining the poly(alkylene glycol) and the alkylene glycol in a stainless steel container and heating the mixture to a temperature of 65±5° C. with stirring (preferably with high shear mixing) for a first period of time (preferably 25-35 minutes, or until homogenized) to obtain a first mixture; (b) slowly adding the polymeric solubilizing agent in small quantities into the first suspension with stirring and continue stirring for a second period of time (preferably 25-35 minutes) after powder addition is complete, at the same temperature to obtain a second mixture; (c) preparing a third mixture by combining the surfactant and acidulant with water in a separate stainless steel container and heating the mixture to a temperature of 65±5° C. with stirring for a third period of time (preferably 12-18 minutes, or until homogenized); (d) combining the second and third mixtures at the same temperature with mixing for a fourth period of time (preferably 13-17 minutes) to provide a fourth mixture; (e) adding the fexofenadine or fexofenadine salt to the fourth mixture in small quantities with stirring at the same temperature and continuing stirring for a fifth period of time (preferably 40-50 minutes) after powder addition has been completed to obtain a fifth mixture; and (f) cooling and deaerating the fifth mixture to ambient temperature, providing the liquid softgel fill composition.

Another aspect of the invention is directed to a softgel capsule comprising a soft gelatin capsule filled with the bioavailable liquid softgel fill composition disclosed above. In one embodiment the gelatin of said soft gelatin (softgel) capsule comprises bovine-, avian-, porcine-, marine- or vegetable-based gelatin, or a mixture of two or more thereof In one embodiment, the softgel capsule further comprises an enteric coating. The enteric coating preferably comprises a controlled release polymer. In one embodiment the controlled release polymer is an acid-resistant polymer.

Another aspect of the invention is directed to a bioavailable liquid softgel fill composition consisting essentially of: a) 4-40% (e.g. 5-35%, 5-20%, 10-30%, 15-25%, about 13% about 14%, about 15% or about 20%) by weight of fexofenadine hydrochloride; b) a matrix comprising: bi) 40-80% (e.g. 45-75%, 50-70%, 55-65%, about 60%, about 64%, 64-65% or about 65%) by weight of PEG 400; bii) 5-30% (e.g. 5-25%, 10-25%, 15-16%, 15-20%, about 14%, about 15% or about 16%) by weight of propylene glycol; biii) 1-10% (e.g. 1-9%, 2-8%, 3-7%, 4-6%, 3-4%, about 3%, about 4% or about 5%) by weight of polyvinylpyrrolidone; and biv) 0.001-6% (e.g., 0.01-2%, 0.1-1%, 0.2-0.5%, about 0.2%, about 0.3% or about 0.4%) by weight of sodium lauryl sulfate; c) 0.001-2% (e.g. 0.01-1%, 0.1-0.8%, 0.4-0.6%, about 0.4%, about 0.5% or about 0.6%) by weight of citric acid; and d) 1-10% (e.g. 1-8%, 2-5%, 2-3%, about 2%, about 2.5% or about 3%) of water, based on the total weight of the composition.

Yet another aspect of the invention is directed to a bioavailable liquid softgel fill composition consisting essentially of: a) about 13.9% by weight of fexofenadine hydrochloride; b) a matrix comprising: bi) about 64.2% by weight of PEG 400; bii) about 15.2% by weight of propylene glycol; biii) about 3.4% by weight of polyvinylpyrrolidone; and biv) about 0.3% by weight of sodium lauryl sulfate; c) about 0.5% by weight of citric acid; and d) about 2.5% of water, based on the total weight of the composition.

One embodiment of the above compositions is directed to a softgel capsule comprising a soft gelatin capsule filled with the above bioavailable liquid softgel fill composition. Preferably the polyvinylpyrrolidone is PVP K17.

The liquid softgel fill formulation can be encapsulated in soft gelatin shells to form softgel capsules using a conventional rotary die process. Suitable soft gelatin shells may include (i) gelatin, 35-60% by weight; (ii) glycerin, 10-15% by weight; (iii) sorbitol, 11-20% by weight; (iv) purified water, 20-40% by weight; and (v) artificial color, 0.0001-0.002% by weight.

The softgel capsules of the invention can also be prepared by other methods well known in the art. See e.g., P. K. Wilkinson et al., “Softgels: Manufacturing Considerations,” Drugs and the Pharmaceutical Sciences, 41 (Specialized Drug Delivery Systems); P. Tyle, Ed. (Marcel Dekker, Inc., New York, 1990) 409-449; F. S. Horn et al., “Capsules, Soft” Encyclopedia of Pharmaceutical Technology, vol. 2; J. Swarbrick and J. C. Boylan, eds. (Marcel Dekker, Inc., New York, 1990) pp. 269-284; M. S. Patel et al., “Advances in Softgel Formulation Technology,” Manufacturing Chemist, vol. 60, no. 7, pp. 26-28 (July 1989); M. S. Patel et al., “Softgel Technology,” Manufacturing Chemist, vol. 60, no. 8, pp. 47-49 (August 1989); R. F. Emerson, “Softgel (Soft Gelatin Capsule) Update,” Drug Development and Industrial Pharmacy (Interphex '86 Conference), vol. 12, no. 8 & 9, pp. 1133-1144 (1986); and W. R. Ebert, “Soft Elastic Gelatin Capsules: A Unique Dosage Form,” Pharmaceutical Technology, vol. 1, no. 5, pp. 44-50 (1977).

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention. The term “about” generally refers to plus or minus 10% of the indicated number. For example, “about 10%” may indicate a range of 9% to 11%, and “about 20” may mean from 18-22. Other meanings of “about” may be apparent from the context, such as rounding off, so, for example “about 1” may also mean from 0.5 to 1.4.

### EXAMPLES

### Example 1

### Bioavailable Fill Composition with Fexofenadine Hydrochloride as Active Ingredient

| Ingredient | Function | Range (wt %) |
|---|---|---|
| PEG 200 to PEG 8000, or | Matrix | 40-80 |
| mixtures | (part A) | |
| Propylene Glycol | | 5-30 |
| Polyvinylpyrrolidone: PVP K17, | Solubilizing | 1-10 |
| PVP K12 PVP K30, PVP K60, | Agent | |
| PVP K90, or mixtures | (Matrix, part B) | |
| Sodium Lauryl Sulfate | | 0.001-6 |
| Water | | 1-10 |
| Citric Acid | Acidulant | 0.001-2 |
| Fexofenadine Hydrochloride | Active Ingredient | 4-40 |

All ingredients were mixed according to the procedure of Example 2.

### Example 2

### Process for Preparation of Fill Composition of Examples 1 and 3

a) Add Propylene Glycol into PEG 400, PEG 600, or other liquid PEG or liquid PEG mixture in a suitable stainless steel container. Heat the solution to 65° C.±5° C. Use a Stainless Steel Propeller/High Shear Mixer to mix all ingredients for 30±5 minutes or until homogenized.
b) Slowly add PVP K17 (or PVP K12, or PVP K30, or PVP K60, or PVP K90, or mixtures) in small quantities to solution a) while continuously mixing at 65° C.±5° C. Mix thoroughly for additional 30±5 minutes after powder addition has been completed.
c) Add Water, Sodium Laryl Sulfate and Citric Acid into a separate suitable stainless steel container. Mix at 65° C.±5° C. for 15±3 minutes or until homogenized.
d) Add Solution c) to Solution b). Continue to mix for additional 15±2 minutes at 65° C.±5° C.
e) Add Fexofenadine HCl into solution d) in small quantities while continuing to mix at 65° C.±5° C. Mix for an additional 45±5 minutes after powder addition has been completed.
f) Cool solution to room temperature and deaerate.

Encapsulate the above suspension into a soft gelatin capsule (softgel). Optionally, the softgel capsules are then provided with an enteric coating consisting of hydroxypropyl methyl cellulose stearate and castor oil as plasticizer, in the customary manner.

### Example 3

### Bioavailable Fill Composition with Fexofenadine Hydrochloride as Active Ingredient

| Ingredient | Function | wt % |
|---|---|---|
| PEG 400 | Matrix | 64.2 |
| Propylene Glycol | (part A) | 15.2 |
| Polyvinylpyrrolidone, PVP K17 | Solubilizing | 3.4 |
| Sodium Lauryl Sulfate | Agent | 0.3 |
| Water | (Matrix, part B) | 2.5 |
| Citric Acid | Acidulant | 0.5 |
| Fexofenadine Hydrochloride | Active Ingredient | 13.9 |

All ingredients were mixed according to the procedure of Example 2.

The specific examples disclosed above are to be construed as merely illustrative, and not limitative of the remainder of the disclosure in any way whatsoever. Without further elaboration, it is believed that one skilled in the art can, based on the description herein, utilize the present invention to its fullest extent.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the present claims.

All publications cited herein are hereby incorporated by reference in their entirety.

1. A bioavailable liquid softgel fill composition comprising:
a) 4-40% by weight of fexofenadine or a fexofenadine salt;
b) a matrix comprising:
i) 40-80% by weight of a pharmaceutically acceptable poly(alkylene glycol);
ii) 5-30% by weight of a pharmaceutically acceptable alkylene glycol;
iii) 1-10% by weight of a pharmaceutically acceptable polymeric solubilizing agent; and
iv) 0.001-0.5% by weight of a pharmaceutically acceptable surfactant; and
c) 0.001-2% by weight of a pharmaceutically acceptable acidulant;
based on the total weight of the composition.

2. The composition of claim 1 which has at least one of the following features:
(a) said fexofenadine or fexofenadine salt and said matrix are present in a ratio of about 1:1.5 to about 1:24 by weight; or
(b) said fexofenadine or fexofenadine salt and said pharmaceutically acceptable polymeric solubilizing agent are present in a ratio of about 40:1 to about 2:5 by weight; or
(c) said fexofenadine or fexofenadine salt and said pharmaceutically acceptable surfactant are present in a ratio of about 40000:1 to about 8:1 by weight.

3. The composition of claim 2, which has all of the features (a), (b) and (c).

4. The composition of claim 1, wherein said fexofenadine salt is fexofenadine hydrochloride.

5. The composition of claim 1, wherein said fexofenadine or fexofenadine salt is the only active ingredient.

6. The composition of claim 1, further comprising one or more additional active ingredients.

7. The composition of claim 1, wherein said pharmaceutically acceptable poly(alkylene glycol) is selected from the group consisting of PEG 200 to PEG 8000, and mixtures thereof.

8. The composition of claim 1, wherein said pharmaceutically acceptable alkylene glycol is propylene glycol.

9. The composition of claim 1, wherein said pharmaceutically acceptable polymeric solubilizing agent is a polyvinylpyrrolidone (PVP).

10. The composition of claim 9, wherein said polyvinylpyrrolidone is selected from the group consisting of PVP K12, PVP K17, PVP K30, PVP K60 and PVP K90.

11. The composition of claim 10, wherein said polyvinylpyrrolidone is PVP K17.

12. The composition of claim 1, wherein said pharmaceutically acceptable surfactant is selected from the group consisting of sodium lauryl sulfate, polysorbates, and PEG-8 caprylic/capric glycerides.

13. The composition of claim 1, wherein said acidulant is selected from the group consisting of pharmaceutically acceptable organic acids and mixtures of two or more thereof.

14. The composition of claim 13, wherein said acidulant is selected from the group consisting of lactic acid, malic acid, citric acid, fumaric acid, ascorbic acid, tartaric acid and mixtures of two or more thereof.

15. The composition of claim 13, wherein said acidulant comprises citric acid.

16. A method of preparing a bioavailable liquid softgel fill composition of claim 1, comprising:
(a) combining the poly(alkylene glycol) and the alkylene glycol in a stainless steel container and heating the mixture to a temperature of 65±5° C. with stirring for a first period of time to obtain a first mixture;
(b) slowly adding the polymeric solubilizing agent in small quantities into the first suspension with stirring for a second period of time at the same temperature to obtain a second mixture;
(c) preparing a third mixture by combining the surfactant and acidulant with water in a separate stainless steel container and heating the mixture to a temperature of 65±5° C. with stirring for a third period of time;
(d) combining said second and third mixtures at the same temperature with mixing for a fourth period of time to provide a fourth mixture;
(e) adding the fexofenadine or fexofenadine salt to the fourth mixture in small quantities with stirring at the same temperature for a fifth period of time to obtain a fifth mixture; and
(f) cooling and deaerating the fifth mixture to ambient temperature, providing the liquid softgel fill composition.

17. A softgel capsule comprising a soft gelatin capsule filled with the bioavailable liquid softgel fill composition of claim 1.

18. The softgel capsule of claim 17, wherein the gelatin of said soft gelatin capsule comprises bovine-, avian-, porcine-, marine- or vegetable-based gelatin, or a mixture of two or more thereof.

19. The softgel capsule of claim 17, further comprising an enteric coating.

20. The softgel capsule of claim 19, wherein said enteric coating comprises a controlled release polymer.

21. The softgel capsule of claim 20, wherein said controlled release polymer is an acid-resistant polymer.

22. A bioavailable liquid softgel fill composition consisting essentially of:
a) 4-40% by weight of fexofenadine hydrochloride;
b) a matrix comprising:
i) 40-80% by weight of PEG 400;
ii) 5-30% by weight of propylene glycol;
iii) 1-10% by weight of polyvinylpyrrolidone; and
iv) 0.001-0.5% by weight of sodium lauryl sulfate;
c) 0.001-2% by weight of citric acid; and
d) 1-10% of water;
based on the total weight of the composition.

23. A bioavailable liquid softgel fill composition consisting essentially of:
a) about 13.9% by weight of fexofenadine hydrochloride;
b) a matrix comprising:
i) about 64.2% by weight of PEG 400;
ii) about 15.2% by weight of propylene glycol;
iii) about 3.4% by weight of polyvinylpyrrolidone; and
iv) about 0.3% by weight of sodium lauryl sulfate;
c) about 0.5% by weight of citric acid; and
d) about 2.5% of water;
based on the total weight of the composition.

24. A softgel capsule comprising a soft gelatin capsule filled with the bioavailable liquid softgel fill composition of claim 23.

25. The fill composition of claim 23, wherein said polyvinylpyrrolidone is PVP K17.

26-27. (canceled)

28. The fill composition of claim 1, wherein said fexofenadine or fexofenadine salt is present in 13-40% by weight.

29. The fill composition of claim 22, wherein said fexofenadine hydrochloride is present in 13-40% by weight.
